(19) United States
(12) Patent Application Publication
(10) Pub. No.: US 2016/0310724 A1
(43) Pub. Date: Oct. 27, 2016

(54) SYSTEM AND METHOD FOR ELECTRODE PLACEMENT IN THE PERICARDIAL SAC OF A PATIENT

(71) Applicant: Guy P. Curtis, San Diego, CA (US)

(72) Inventor: Guy P. Curtis, San Diego, CA (US)

(21) Appl. No.: 14/815,711

(22) Filed: Jul. 31, 2015

Related U.S. Application Data
(63) Continuation-in-part of application No. 14/695,237, filed on Apr. 24, 2015.

(51) Int. Cl. A61N 1/05

(52) U.S. Cl. CPC A61N 1/059

(57) ABSTRACT

A device for stabilizing an electrode adjacent to a sympathetic nerve of a patient includes an elongated placement catheter having an electrode mounted at its distal end. The placement catheter is dimensioned for advancement through the potential space between the myocardium and the pericardial sac around the heart muscle of the patient, to position the electrode adjacent the sympathetic nerve. When activated, an engagement mechanism on the placement catheter inserts an anchor into the myocardium to stabilize and maintain the location of the electrode relative to the sympathetic nerve.

This application is a continuation-in-part of application Ser. No. 14/695,237, filed Apr. 24, 2015, which is currently pending. The contents of application Ser. No. 14/695,237 are incorporated herein by reference.

### FIELD OF THE INVENTION

The present invention pertains to systems and methods for improving heart muscle function. More particularly, the present invention pertains to systems and methods which stimulate sympathetic nerves to secrete norepinephrine during the absolute refractory period of a heart muscle cycle, to thereby improve heart muscle contraction. The present invention is particularly, but not exclusively, useful as a system or method wherein nerve stimulation in the absolute refractory period is triggered by a local electrical depolarization of the heart muscle.

### BACKGROUND OF THE INVENTION

A normal heart muscle cycle (i.e. a heartbeat) is repetitive and is characterized by several well-known and distinctly identifiable mechanical and electrical characteristics. In its mechanical cycle, the heart muscle alternately functions to pump blood into the vasculature of a patient by its contractions (systole), and to receive blood from the vasculature by its relaxation (diastole). Physiologically, the heart muscle cycle is the result of an electrical cycle that is superposed on the mechanical cycle. Of immediate interest here, however, is the absolute refractory period.

During the absolute refractory period, which follows cell firing during diastole, and which is approximately 120-300 msec in duration, the heart muscle is not able to respond to an electrical stimulation. Sympathetic nerves on the epicardial surface of the heart, however, can be electrically stimulated during the absolute refractory period to thereby secrete norepinephrine. The import here is that the secreted norepinephrine can then subsequently assist in controlling and improving a heart muscle contraction. It is, of course, essential to stimulate the sympathetic nerve during the heart's absolute refractory period so that the electrical and mechanical cycles of the heart are not disturbed.

Identifying the appropriate time for electrically stimulating a sympathetic nerve must necessarily be established relative to the heart muscle cycle. Heretofore, the timing for nerve stimulation has been determined by the operation of a pacing device. For example, U.S. Pat. No. 8,463,376, which issued to Curtis for an invention entitled “System and Method for Transvascular Activation of Cardiac Nerves with Automatic Restart,” discloses and claims the electrically paced stimulation of a heart muscle.

The present invention now recognizes that the heart muscle itself creates natural signals (i.e. electrical events) which can be used to trigger a subsequent electrical stimulation of a sympathetic nerve. Importantly, this subsequent nerve stimulation can be timed to occur in the absolute refractory period of the heart muscle cycle.

To be effective, an electrode must be stabilized when it is used for the purpose of stimulating a sympathetic nerve to assist with a contraction of the patient's heart muscle. As disclosed elsewhere here, the required stabilization is provided for this purpose when the electrode is positioned in an epicardial vein, adjacent a sympathetic nerve, on the heart's epicardial surface. Other approaches for a proper electrode placement, however, are also anatomically possible. Of specific interest here is the potential space that exists between the myocardium and the pericardial sac.

With the above in mind, it is an object of the present invention to provide a system and method for electrically stimulating a sympathetic nerve of a patient in response to a naturally occurring electrical event of the heart muscle. Another object of the present invention is to electrically stimulate a sympathetic nerve of a patient using a single pulse, or multiple pulses, during the absolute refractory period of a heart muscle cycle to assist with a contraction of the patient's heart muscle. Yet another object of the present invention is to stabilize an electrode in a position adjacent a sympathetic nerve to prevent electrode movements that could otherwise cause pericarditis or irritations due to possible tissue damage. Still another object of the present invention is to provide a system and method for electrically stimulating a sympathetic nerve of a patient which is easy to use, is simple to manufacture and is commercially cost effective.

### SUMMARY OF THE INVENTION

In accordance with the present invention, a system and method are provided to improve the heart contractions of a patient during a heart function cycle (heartbeat). To set up the system for its operation, a deployment catheter is used to position an electrode and a sensor in an epicardial vein that is located on the epicardial surface of the heart. A proper positioning of the electrode and the sensor requires they be located adjacent a sympathetic nerve.

In an overview of the present invention, it is to be appreciated that, during each heart function cycle, the present invention detects a local electrical event (depolarization) of the heart muscle. Based on the occurrence of this local electrical event at a time t₀, a stimulation interval, Δt, is established. In detail, Δt begins at the time t₀, and it ends at a time t₁ during the absolute refractory period of the heart function cycle. At the time t₁, the sympathetic nerve, which is located on the epicardial surface of the heart, is stimulated. With this stimulation the sympathetic nerve will secrete norepinephrine to improve a subsequent contraction of the heart.

Structurally, a device of the present invention includes a sensor for detecting the local electrical event of the heart, at the time t₀. Typically, a local electrical event is selected and used that occurs during a heart contraction, during diastole, in the patient's natural heart muscle cycle. The device also includes a timer that is activated at the time t₀ and is used for measuring the predetermined stimulation interval Δt. Recall, Δt extends between the start time t₀ and the time t₁ in the absolute refractory period of the patient's heart cycle (Δt=t₁−t₀). Further, the device includes a stimulator that is connected with an electrode for stimulating the sympathetic nerve with at least one electrical pulse at the time t₁. Preferably, the electrical pulse(s) for stimulating the sympathetic nerve has(have) a predetermined intensity that is essentially less than about three times the intensity required for activating a contraction of the heart muscle.

For an alternate embodiment of the present invention, the system of the present invention may also include a pacing device which, along with the sensor, can be selectively connected by a switch with the stimulator. For this embodiment, when selected, the pacing device is used to electronically establish to. Although the switch can be used to selectively alternate between a connection of the stimulator with the sensor, or with the pacing device, the overall purpose and functionality of the system remains unchanged.

Additional components for the device of the present invention include a voltage source that will generate the electrical pulse at the time ti. These components also include a computer for coordinating an operation of the stimulator with respective operations of the sensor, the pacing device, the switch, and the timer.

From a functional perspective, the methodology of the present invention is dependent on the heart function cycle. Accordingly, a method for electrically stimulating a sympathetic nerve of a patient to improve heart function requires first positioning an electrode/sensor in an epicardial vein, on the epicardial surface of the heart, adjacent the sympathetic nerve. The electrode/sensor is then used to detect a local electrical event. Specifically, the local electrical event that is to be detected by the sensor needs to occur near the electrode and will result from the patient's natural heart muscle cycle.

Once a local electrical event is detected, a computer can then be used to establish a predetermined stimulation interval Δt that will extend from the start time t₀, to a time t₁. As noted above, the time t₁ needs to fall in the absolute refractory period of the patient's natural heart muscle cycle (t₁−t₀=Δt). The computer can then activate the stimulator at the time The purpose here, of course, is to electrically stimulate the sympathetic nerve with at least one electrical pulse, to thereby improve a subsequent contraction of the patient's heart muscle.

In another aspect of the present invention, a device and method are provided for accessing the sympathetic nerve to be stimulated by using a different approach. Specifically, the present invention also envisions approaching the sympathetic nerve through the potential space between the myocardium and the pericardial sac surrounding the heart muscle.

For this aspect of the invention, an elongated placement catheter is provided which has an electrode mounted at its distal end. The placement catheter is dimensioned for insertion into the potential space between the myocardium and the pericardial sac. Thus, the electrode on the placement catheter is advanced through the potential space to a position adjacent the sympathetic nerve. Once the electrode has been positioned at a proper location, an engagement mechanism which is located on the placement catheter is activated to insert an anchor into the myocardium, to thereby stabilize and maintain the location of the electrode relative to the sympathetic nerve.

In a preferred embodiment of the placement catheter, the catheter is formed with a hollow lumen that is surrounded by an outer wall. For this embodiment, the engagement mechanism includes a threaded shaft having an end that is fixedly attached to the wall. As attached, the shaft extends diametrically across the lumen inside the placement catheter between its attachment point and an aperture that is formed into the wall of the catheter opposite the attachment point. Also included in the preferred embodiment is a bar which is formed with a serrated surface. Specifically, the bar is mounted in the lumen of the placement catheter for axial movement in the catheter's lumen.

Along with the threaded shaft and the serrated bar, an actuator is also included in the preferred embodiment of the engagement mechanism. In detail, the actuator is a hollow, cylindrically shaped body that is formed with an internal cylindrical surface and it has an external cylindrical surface that is parallel to the internal surface. Importantly, the internal surface is formed for a threaded engagement with the threaded shaft and the external surface is serrated for engagement with the serrated surface of the bar.

For the preferred embodiment, a corkscrew is used as the anchor. Specifically, the corkscrew is affixed to the actuator for extension of the corkscrew through the aperture in the wall of the placement catheter. Thus, when the bar is pulled in a proximal direction, the actuator is rotated for advancement along the threaded shaft. This then extends the corkscrew from the placement catheter and into engagement with the myocardium to thereby stabilize the electrode against the myocardium.

In an alternate embodiment of the placement catheter, the anchor is a prong. For this alternate embodiment, the engagement mechanism is formed with a compartment inside the placement catheter. The prong is then mounted in the compartment for movement between a first configuration wherein the prong is withdrawn into the compartment, and a second configuration wherein the prong extends from the compartment. More specifically, to move the prong into the second configuration it is effectively rotated on the placement catheter to extend from the catheter and into the myocardium to stabilize the electrode against the myocardium.

In detail, for this alternate embodiment of the present invention, the compartment is formed with an opening and it has an arcuate surface that extends from the opening to an abutment that is formed inside the compartment. Also, the prong is formed with a locking pin. Further, the engagement mechanism includes a push rod that is engaged with the prong by a pivot pin to permit a rotation of the prong on the pivot pin. In this combination, when the push rod is advanced in a distal direction on the placement catheter several functions occur simultaneously. For one, the prong is urged against the arcuate wall of the compartment. This, in turn, causes the prong to rotate on the pivot pin and into its second configuration. Further, with this rotation of the prong the locking pin of the prong is positioned against the abutment in the compartment. The prong is thereby secured in the compartment in its second configuration. Moreover, as it rotates, the prong is inserted into the myocardium to thereby stabilize the electrode against the myocardium.

Additional considerations for both of the embodiments disclosed above include the preference that in each instance the anchor will extend from the placement catheter through a distance less than ⅛ inch. Also, both embodiments will include an orientation mark that is located at the distal end of the placement catheter. The purpose here is to observe the mark for verifying a proper positon and orientation of the placement catheter prior to stabilizing the electrode. Functionally, the orientation mark should be observable only within an arc of less than 180°. This function can be performed by any method well known in the pertinent art, such as by using systems which incorporate a technology such as OCT, MRI, or fluoroscopy.

A method for employing the devices disclosed above will include the steps of advancing the electrode on the placement catheter through the potential space between the myocardium and the pericardial sac around the heart muscle. Next, a user of the present invention needs to orient the electrode at a location adjacent the sympathetic nerve of the patient that is to be stimulated. Once the electrode has been oriented at a proper location, the engagement mechanism on the placement catheter is activated to insert the anchor into the myocardium to stabilize and maintain the location of the electrode relative to the sympathetic nerve.

With the above in mind, it is to be understood that an operation of the present invention requires consecutively repeating the stimulation interval Δt for each heart function cycle. Also, the stimulation interval Δt that determines when a sympathetic nerve is to be stimulated is in an approximate range of 100-120 msec. Further, the electrical pulse(s) for stimulating the sympathetic nerve has(have) a predetermined intensity that is less than about three times the intensity required for activating a contraction of the heart muscle.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a depiction of a system in accordance with the present invention shown together with the intended environment of its operation;

FIG. 2 is a functional layout of the components employed in a system of the present invention;

FIG. 3 is a time-line depiction of a heart muscle cycle with an operation of the present invention superposed thereon in its relation to the absolute refractory period;

FIG. 4 is a logic flow chart for the functional tasks that are required during an operation of the computer-controlled system of the present invention;

FIG. 5 is a perspective view of the distal end of a placement catheter of the present invention;

FIG. 6A is a cross-section view of an anchor (i.e. a corkscrew) for the preferred embodiment of the present invention as seen along the line 6/8-6/8 in FIG. 5, prior to a deployment of the anchor from the placement catheter;

FIG. 6B is a cross-section view of the anchor for the preferred embodiment of the present invention as shown in FIG. 6A, after a deployment of the anchor from the placement catheter;

FIG. 7 is a plan cross-section view of an anchor (i.e. a prong) as seen along the line 7-7 in FIG. 5;

FIG. 8A is a cross-section view of the anchor (i.e. a prong) for the alternate embodiment of the present invention as seen along the line 6/8-6/8 in FIG. 5, prior to a deployment of the anchor from the placement catheter; and

FIG. 8B is a cross-section view of the anchor for the alternate embodiment of the present invention as shown in FIG. 8A, after a deployment of the anchor from the placement catheter.

### DESCRIPTION OF THE PREFERRED EMBODIMENTS

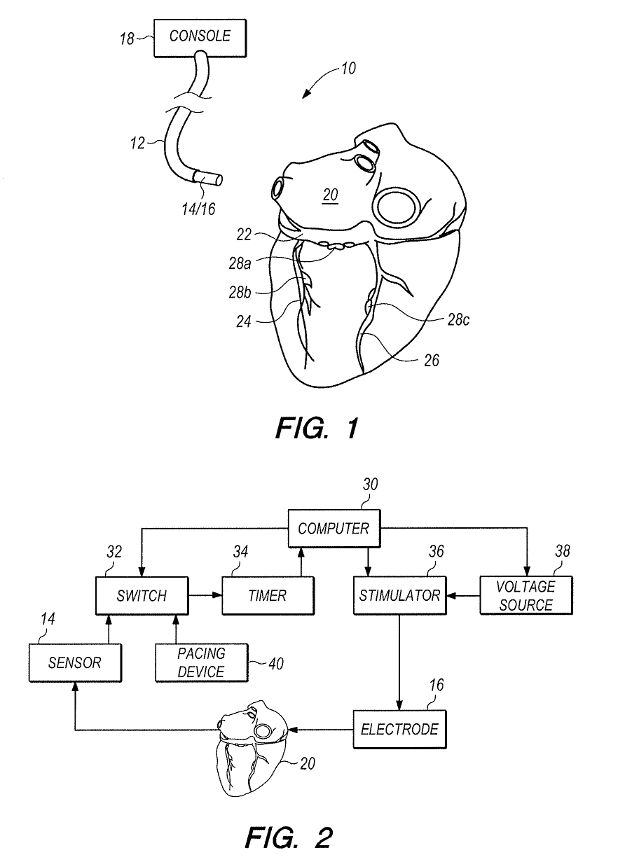

Referring initially to FIG. 1, a system for electrically stimulating a sympathetic nerve of a patient to improve heart function is shown and is generally designated 10. As shown, the system 10 includes a deployment catheter 12 which has a sensor 14 and an electrode 16 that are mounted in combination at the distal end of the deployment catheter 12. In addition to the mechanical components mentioned above, the system 10 also includes various electronic components which are disclosed below with reference to FIG. 2. As disclosed below, these electronic components are mounted in the console 18 and interact with each other to provide operational control over the system 10. For purposes of the present invention, it is to be appreciated that the console 18 may be either extracorporeal or implantable. For example, as an implantable, the console 18 may be part of a pacemaker or a defibrillator.

Still referring to FIG. 1, a heart muscle 20 is shown as the surgical target for the present invention. Anatomically, a view of the diaphragmatic surface of the heart muscle 20 shows its coronary sinus 22 and several connecting veins. In particular, the posterior vein 24 of the left ventricle, and the middle cardiac vein 26 are shown. Also shown are sympathetic nerve(s) 28 in the nervous system, of which the nerve bundles 28a, 28b and 28c are only exemplary. Importantly, the nerves 28 are located on the epicardial surface of the left ventricle, and they are adjacent to either the coronary sinus 22 or one of the veins connected with the coronary sinus 22 (e.g. veins 24 or 26).

Referring now to FIG. 2, it will be seen that a computer 30 is provided for the system 10, and that the computer 30 is electronically connected with a switch 32, a timer 34, a stimulator 36 and a voltage source 38. Optionally, a pacing device 40 can also be electronically incorporated with the aforementioned components. As will be best appreciated by cross-reference between FIG. 1 and FIG. 2, the switch 32, the timer 34, the stimulator 36, and the voltage source 38, as well as the pacing device 40, can all be mounted on the extracorporeal console 18. On the other hand, as disclosed above, the sensor 14 and the electrode 16 are incorporated into the deployment catheter 12.

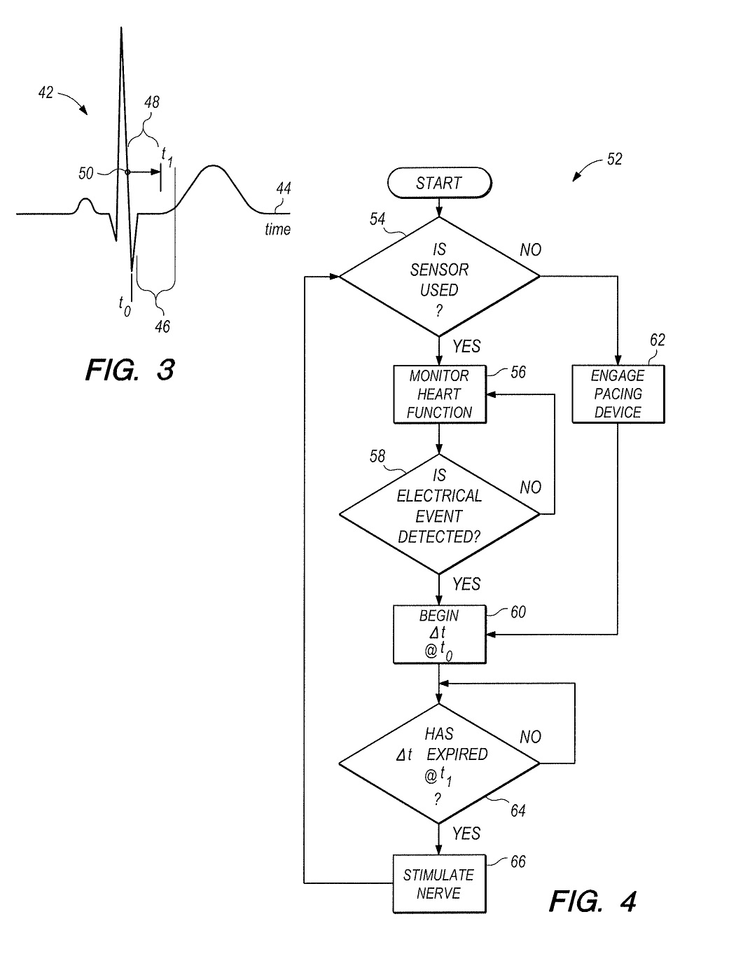

For a disclosure of their interaction with each other, the components mentioned above are shown in FIG. 2 in their relationship with the heart muscle 20. Specifically, the sensor 14 and the electrode 16 are operationally shown in direct contact with the heart muscle 20. Depending on whether the operation of system 10 is to rely on a paced event, which can be alternatively provided using the pacing device 40, the switch 32 is used to alternatively connect the sensor 14 or the pacing device 40 with the timer 34. Further, under the control of the computer 30, the stimulator 36 is energized by the voltage source 38 for a timed activation of the electrode 16. In turn, the electrode 16 will stimulate a sympathetic nerve 28 on the heart muscle 20. As envisioned for the present invention, each pulse that is used to stimulate the sympathetic nerve 28 will have an intensity that is less than about three times the intensity required for activating a contraction of the heart muscle 20. The depiction of a normal heart function cycle (i.e. a heartbeat) is shown in FIG. 3 and is generally designated 42. As shown, the heart function cycle 42 is depicted by an isoelectric line 44. In this context, the absolute refractory period 46 of the heart function cycle 42 is shown in its overall relationship with the heart function cycle 42. As discussed above, the absolute refractory period 46 is a period of time in which the heart muscle 20 is not able to respond to an electrical stimulation. As also discussed above, the present invention requires there be a stimulation of a sympathetic nerve 28 during the absolute refractory period 46. To do this, the system 10 of the present invention establishes a stimulation interval 48 that will begin with an electrical event 50 at a time t₀ and will end at a time t₁ in the absolute refractory period 46 when a sympathetic nerve 28 is stimulated.

Still referring to FIG. 3 an exemplary electrical event 50 is shown on the isoelectric line 44 to occur at a time t₀. As envisioned by the present invention, the exact time for selection of an occurrence for the electrical event 50 is somewhat arbitrary. Preferably, however, it will be before and relatively near the beginning of the absolute refractory period 46. As indicated above, in an alternate embodiment of the present invention a pacing device 40 can be employed to set the start time t₀. In any event, once a time t₀ has been determined for the electrical event 50, or set by the pacing device 40, the stimulation interval 48 can be established. Mathematically expressed, t₁−t₀=Δt, wherein Δt is the stimulation interval 48. Preferably, Δt will be in an approximate range of 100 to 120 msec. Again, note with reference to FIG. 3 that the time t₁ falls within the absolute refractory period 46.

A logic flow chart for the tasks to be performed during an operation of the present invention is shown in FIG. 4 and is generally designated 52. After the start of an operation, the inquiry block 54 questions whether the sensor 14 is being used. If so, task block 56 requires that the heart muscle function be monitored by the sensor 14. Next, inquiry block 58 asks whether an electrical event 50 has been detected. If not, the sensor 14 continues monitoring the heart function cycle 42. On the other hand, if an electrical event 50 is detected, task block 60 requires the establishment of a stimulation interval 48.

As disclosed above, the stimulation interval Δt 48 extends from a time t₀ when the electrical event 50 is detected, to a time t₁ when a pulse(s) is(are) to be fired by the stimulator 36 to stimulate a sympathetic nerve 28. Recall, in an alternate embodiment of the present invention, a pacing device 40, rather than the sensor 14, is used to trigger the stimulation interval 48. Thus, for the alternate embodiment, inquiry block 54 together with task block 62 directs there be an engagement of the timer 34 with the pacing device 40. In all embodiments, however, the inquiry block 64 and task block 66, together, indicate that when the stimulation interval 48 has expired, the stimulator 36 is activated by the computer 30 to stimulate the sympathetic nerve 28. The system 10 then proceeds to monitor the next heart function cycle 42.

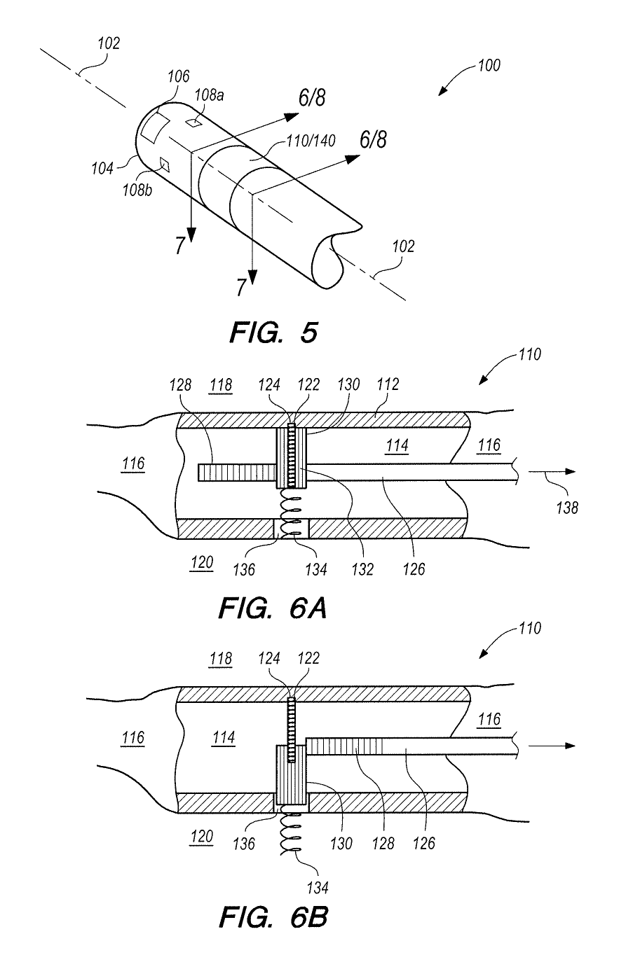

Referring now to FIG. 5, a portion of a placement catheter for use in another aspect of the present invention is shown and is generally designated 100. As shown, the placement catheter 100 defines a longitudinal axis 102 and has a distal end 104. An electrode 106 is mounted on the distal end 104 along with a pair of identification markers 108a and 108b. As will be appreciated with the disclosure presented below, only one of the identification markers 108 may suffice for purposes of placing the placement catheter 100. FIG. 1 also shows that an engagement mechanism 110 is provided for the present invention, and that the engagement mechanism 110 is positioned on the placement catheter 100 just proximal the distal end 104. Details of the engagement mechanism 110 are best appreciated with reference to FIG. 6A.

In FIG. 6A it is seen that the engagement mechanism 110 includes a portion of the outer wall 112 of the placement catheter 100 which surrounds a lumen 114. As further indicated in FIG. 6A, the present invention envisions use of the placement catheter 100, and the engagement mechanism 110, in the potential space 116 which is located between the pericardial sac 118 and the myocardium 120 of a heart muscle 20.

Still referring to FIG. 6A, it is seen that the engagement mechanism 110 includes a threaded shaft 122 which is attached to the outer wall 112 of the placement catheter 110 at an attachment point 124. Specifically, with this attachment, the threaded shaft 122 is diametrically oriented in the lumen 114. Also included in the engagement mechanism 110 is a rod 126 which has a serrated surface 128. Further, engagement mechanism 110 includes an actuator 130 which has a serrated surface 132 that is engaged with the serrated surface 128 of the rod 126. Additionally, an anchor 134 (i.e. a corkscrew) is mounted on the actuator 130 and, as shown, is positioned over an aperture 136 which extends through the outer wall 112 of the placement catheter 100.

With the combination of components disclosed above for the engagement mechanism 110, an operation of the engagement mechanism 110 merely requires pulling on the rod 126 in a proximal direction as indicated by arrow 138 in FIG. 6A. In the operation, the proximal movement of rod 126 will rotate the actuator 130. This rotation of the actuator 130 will, in turn, cause the actuator 130 to advance along the threaded shaft 122 toward the aperture 136. Thus, the anchor 134 (corkscrew) is also rotated as it extends from the placement catheter 100 through the aperture 136. As intended for the present invention, the anchor 134 will extend from the placement catheter 100 through a distance less than about ⅛ of an inch as it engages with the myocardium 120 (see FIG. 6B) to stabilize and maintain the location of the electrode 106.

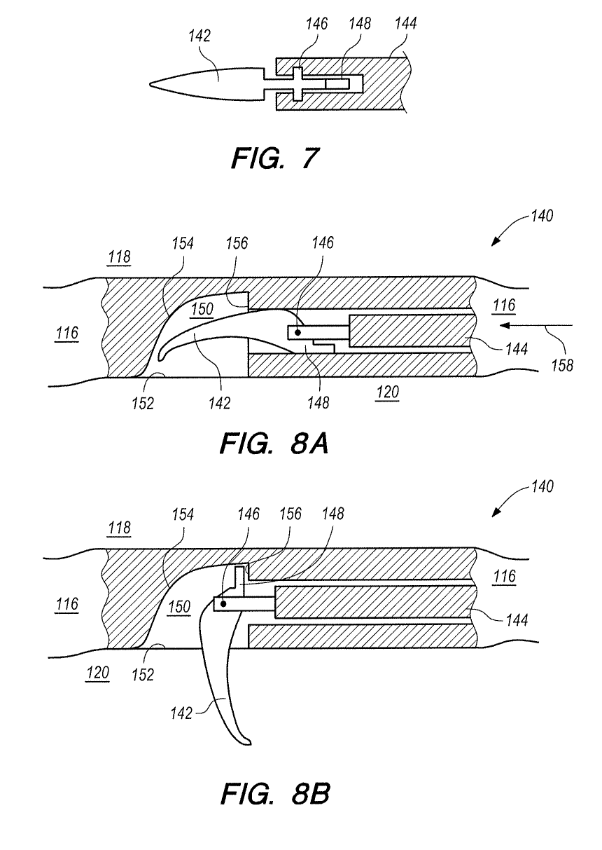

For an alternate embodiment of the present invention, FIG. 7 shows an assembly of components that are to be incorporated into an engagement mechanism 140. As will be best appreciated by cross-referencing FIG. 7 with FIGS. 8A and 8B, the engagement mechanism 140 includes and anchor 142 (i.e. prong) which is engaged with a push rod 144 by a pivot pin 146. It is also to be noted that the anchor 142 is formed with a locking pin 148. With specific reference to FIGS. 8A and 8B, it will be seen that for the engagement mechanism 140 the placement catheter 100 is formed with a compartment 150 having an exit opening 152. As shown, the compartment 150 has an arcuate surface 154 that extends from the opening 152 to an abutment 156.

In an operation of the engagement mechanism 140, the push rod 144 is advanced in a distal direction as indicated by the arrow 158 in FIG. 8A. With this advancement, the anchor (prong) 142 is guided in rotation about the pivot pin 146 by the arcuate surface 154. This advancement continues until the locking pin 148 on the anchor (prong) 142 has entered the compartment 150. The locking pin 148 can then be brought into contact with the abutment 156 to hold the anchor (prong) 142 in an extended configuration as shown in FIG. 8B.

As intended for the present invention, and similar to the preferred embodiment disclosed above with reference to FIGS. 6A and 6B, the anchor (prong) 140 will extend from the placement catheter 100 through a distance less than about ⅛ of an inch as it engages with the myocardium 120 (see FIG. 8B) to stabilize and maintain the location of the electrode 106.

While the particular System and Method for Electrode Placement in the Pericardial Sac of a Patient as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for stabilizing an electrode adjacent to a sympathetic nerve of a patient to stimulate the nerve for improved heart muscle function which comprises:
an elongated placement catheter having a proximal end and a distal end, with the electrode mounted at the distal end of the placement catheter, wherein the placement catheter is dimensioned for insertion into the potential space between the myocardium and the pericardial sac around the heart muscle, and for advancement of the placement catheter through the potential space to position the electrode adjacent a sympathetic nerve;
an engagement mechanism located on the placement catheter adjacent the electrode; and
a means for activating the engagement mechanism to insert an anchor therefrom into the myocardium to stabilize and maintain the location of the electrode relative to the sympathetic nerve.

2. A device as recited in claim 1 wherein the placement catheter is formed with a hollow lumen surrounded by an outer wall, and the engagement mechanism comprises:
a threaded shaft having a first end and a second end, wherein the first end of the shaft is fixedly attached to the wall of the placement catheter to extend the shaft diametrically across the lumen inside the placement catheter from its attachment point toward an aperture formed in the wall opposite the attachment point;
a bar formed with a serrated surface mounted in the lumen of the placement catheter for axial movement in the lumen between the proximal end and the distal end of the catheter;
an actuator formed as a hollow cylindrical shaped body with an internal cylindrical surface and an external cylindrical surface, wherein the internal surface is parallel to the external surface, and wherein the internal surface is formed for a threaded engagement with the threaded shaft and the external surface is serrated for engagement with the serrated surface of the bar; and
a corkscrew used as the anchor, wherein the corkscrew is affixed to the actuator for extension of the corkscrew through the aperture in the wall of the placement catheter and into engagement with the myocardium, when the bar is moved in a proximal direction through the lumen of the placement catheter to rotate the actuator for advancement thereof along the threaded shaft to extend the corkscrew from the placement catheter and to stabilize the electrode against the myocardium.

3. A device as recited in claim 1 further comprising an orientation mark located at the distal end of the placement catheter for use in verifying a proper positon and orientation of the placement catheter prior to stabilizing the electrode.

4. A device as recited in claim 3 wherein the orientation mark is observable within an arc of less than 180°.

5. A device as recited in claim 4 wherein the orientation mark is observable by a system incorporating a technology selected from the group consisting of OCT, MRI, and fluoroscopy.

6. A device as recited in claim 1 wherein the anchor extends from the placement catheter through a distance less than ⅛ inch.

7. A device as recited in claim 1 wherein the anchor is a prong and the engagement mechanism is formed with a compartment inside the placement catheter, and wherein the prong is mounted on the placement catheter for movement between a first configuration wherein the prong is withdrawn into the compartment inside the placement catheter and a second configuration wherein the prong is moved from the compartment and is rotated on the placement catheter to extend the prong therefrom to stabilize the electrode against the myocardium.

8. A device as recited in claim 7 wherein the compartment is formed with an opening and has an arcuate surface extending from the opening to an abutment formed inside the compartment, wherein the prong is formed with a locking pin, and wherein the engagement mechanism further comprises:
a push rod;
a pivot pin mounted on the push rod and engaged with the prong for rotation of the prong on the pivot pin; and
a means for advancing the push rod in a distal direction on the placement catheter to simultaneously urge the prong against the arcuate wall of the compartment, to rotate the prong into its second configuration, and to position the locking pin of the prong against the abutment of the compartment to secure the prong in the compartment in its second configuration and stabilize the electrode against the myocardium.

9. A method for stabilizing an electrode adjacent to a sympathetic nerve of a patient to stimulate the nerve for improved heart muscle function which comprises the steps of:
advancing an electrode on a placement catheter through a potential space between the myocardium and the pericardial sac around the heart muscle to a location adjacent a sympathetic nerve of the patient;
orienting the electrode at the location; and
activating an engagement mechanism on the placement catheter to insert an anchor therefrom into the myocardium to stabilize and maintain the location of the electrode relative to the sympathetic nerve.

10. A method as recited in claim 9 wherein the anchor is a corkscrew mounted on an actuator, and the placement catheter is formed with a hollow lumen surrounded by an outer wall, and also wherein the engagement mechanism includes a threaded shaft having a first end and a second end with the first end of the shaft fixedly attached to the wall of the placement catheter to extend the shaft diametrically across the lumen inside the placement catheter from its attachment point toward an aperture formed in the wall opposite the attachment point, and further wherein a bar formed with a serrated surface is mounted in the lumen of the placement catheter for axial movement in the lumen between the proximal end and the distal end of the catheter, and still further wherein the actuator is formed with an internal cylindrical surface and a parallel external surface, with the internal surface formed for a threaded engagement with the threaded shaft and the external surface serrated for engagement with the serrated surface of the bar, and wherein the method further comprises the steps of:
pulling the bar in a proximal direction through the lumen of the placement catheter to rotate the actuator for advancement thereof along the threaded shaft to extend the corkscrew from the placement catheter; and
ceasing the pulling step when the corkscrew has been extended through a distance less than ⅛ inch from the placement catheter to stabilize the electrode against the myocardium.

11. A method as recited in claim 9 wherein the anchor is a prong and the engagement mechanism is formed with a compartment inside the placement catheter, and wherein the prong is mounted on the placement catheter for movement between a first configuration inside the compartment and a second configuration with the prong extended from the placement catheter, and wherein the method further comprises the steps of:
pushing on the prong in a distal direction to rotate the prong on the placement catheter from the first configuration to the second configuration; and
ceasing the pushing step when the prong has been extended through a distance less than ⅛ inch from the placement catheter to stabilize the electrode against the myocardium.

12. A method as recited in claim 9 wherein an orientation mark is located at the distal end of the placement catheter for use in verifying a proper positon and orientation of the placement catheter, and the orienting step is accomplished by observing the orientation mark within an arc of less than 180° prior to stabilizing the electrode.
